(19) United States
(12) Patent Application Publication
(10) Pub. No.: US 2012/0078197 A1
(43) Pub. Date: Mar. 29, 2012

(54) PORTABLE INFUSION PUMP WITH ANTI-SIPHONING PROTECTION

(76) Inventors: Sean O'Connor, West Chester, PA (US); Luis Jahn, Royersford, PA (US)

(21) Appl. No.: 13/239,502

(22) Filed: Sep. 22, 2011

Related U.S. Application Data
(60) Provisional application No. 61/386,163, filed on Sep. 24, 2010.

(51) Int. Cl. A61M 5/168

(52) U.S. Cl. 604/247

(57) ABSTRACT

Described is drug infusion device with one or more check valves for inhibiting the unintentional discharge of medication from a cartridge. The device includes a chamber capable of receiving a cartridge of medication in a fluid form and one or more novel check valves for ensuring the drug is not unintentionally released due to pressure differentials between the cartridge and the ambient pressure outside of the drug infusion device.

### CROSS REFERENCE TO RELATED APPLICATIONS

This application relates to U.S. patent application Ser. No. 61/386,163, filed Sep. 24, 2010; all applications are herein incorporated by reference in their entireties.

### FIELD OF THE INVENTION

The present invention relates, in general, to drug delivery devices and, more particularly, to systems and methods for detecting pressure differentials in portable drug infusion devices and inhibiting the unintentional discharge of drug due to pressure changes and siphoning.

### BACKGROUND OF THE INVENTION

The use of drug delivery devices for various types of drug therapy is becoming more common as the automated infusion of a drug may provide more reliable and more precise treatment to a patient.

Diabetes is a major health concern, as it can significantly impede on the freedom of action and lifestyle of persons afflicted with this disease. Typically, treatment of the more severe form of the condition, Type I (insulin-dependent) diabetes, requires one or more insulin injections per day, referred to as multiple daily injections. Insulin is required to control glucose or sugar in the blood, thereby preventing hyperglycemia that, if left uncorrected, can lead to ketosis. Additionally, improper administration of insulin therapy can result in hypoglycemic episodes, which can cause coma and death. Hyperglycemia in diabetics has been correlated with several long-term effects of diabetes, such as heart disease, atherosclerosis, blindness, stroke, hypertension, and kidney failure.

The value of frequent monitoring of blood glucose as a means to avoid or at least minimize the complications of Type I diabetes is well established. Patients with Type II (non-insulin-dependent) diabetes can also benefit from blood glucose monitoring in the control of their condition by way of diet and exercise. Thus, careful monitoring of blood glucose levels and the ability to accurately and conveniently infuse insulin into the body in a timely manner is a critical component in diabetes care and treatment.

To more effectively control diabetes in a manner that reduces the limitations imposed by this disease on the lifestyle of the affected person, various devices for facilitating blood glucose (BG) monitoring have been introduced. Typically, such devices, or meters, permit the patient to quickly, and with a minimal amount of physical discomfort, obtain a sample of their blood or interstitial fluid that is then analyzed by the meter. In most cases, the meter has a display screen that shows the BG reading for the patient. The patient may then dose theirselves with the appropriate amount, or bolus, of insulin. For many diabetics, this results in having to receive multiple daily injections of insulin. In many cases, these injections are self-administered.

Due to the debilitating effects that abnormal BG levels can have on patients, i.e., hyperglycemia, persons experiencing certain symptoms of diabetes may not be in a situation where they can safely and accurately self-administer a bolus of insulin. Moreover, persons with active lifestyles find it extremely inconvenient and imposing to have to use multiple daily injections of insulin to control their blood sugar levels, as this may interfere or prohibit their ability to engage in certain activities. For others with diabetes, multiple daily injections may simply not be the most effective means for controlling their BG levels. Thus, to further improve both accuracy and convenience for the patient, insulin infusion pumps have been developed.

Insulin pumps are generally devices that are worn on the patient's body, either above or below their clothing. Because the pumps are worn on the patient's body, a small and unobtrusive device is desirable. Some devices are waterproof, to allow the patient to be less inhibited in their daily activities by having to remove their drug infusion device while showering, bathing, or engaging in various activities that might subject their infusion device to moister, such as swimming. In such devices, it would be desirable to have a structure and method for verifying proper function of venting system within the device, since vents are typically passive devices that have no means for self-diagnostic checks to verify function has been compromised (i.e. intentional or unintentional obstruction of vent opening(s)). Further, it would be desirable to be able to alert the user of abnormal pressure differentials within their device that may cause erratic or unintentional drug delivery. Finally, it would be desirable for a drug infusion device to incorporate means for detecting the altitude at which the device is located, to avoid problems associated with air travel and sporting activities such as mountain climbing, skydiving, etc. that patients may wish to engage in without having to forego the use of their drug infusion device for concerns over erratic or unintentional drug delivery due to rapid pressure changes in and around the device.

Further, it would be desirable for a portable infusion pump to have means to inhibit the unintended discharge of drug caused by pressure differentials and siphoning, as it has been a longstanding problem in the art that these phenomena may occur when the pressure outside of a drug-containing reservoir decreases below the pressure inside of the reservoir. This problem is particularly notable for insulin-dependent diabetic who must disconnect and remove their portable insulin pumps during air travel to avoid accidental and potentially harmful overdosing of medication.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary embodiment of a drug infusion device according to the present invention, in cross-section.

FIG. 2 illustrates another exemplary embodiment of a drug infusion device according to the present invention in exploded view.

FIG. 3 illustrates another exemplary embodiment of a drug infusion device according to the present invention in perspective view and partly in cross-section.

### DETAILED DESCRIPTION OF ILLUSTRATIVE EMBODIMENTS OF THE INVENTION

In an exemplary embodiment, the invention is directed to structures and methods for avoiding the accidental or unintentional discharge of medication from a portable drug infusion device caused by pressure differentials between the compartment that houses the drug reservoir of a portable drug infusion pump and the external environment (atmosphere).

Some portable infusion pumps are designed to be waterproof. This is an attractive feature for people with active lifestyles who benefit from continuous drug infusion (i.e. infusion of insulin for people with diabetes). Such devices must be designed with sealed enclosures/housings to prevent ingress of water. To avoid the development of pressure differentials between the external environment and the sealed compartment that houses the drug reservoir, most waterproof pumps incorporate hydrophobic vents that allow passage of air, but not fluids (within certain limitations of pressure differential).

Most portable drug infusion pump reservoirs are similar in design to that of a standard syringe. Therefore, the reservoir is typically comprised of two major components; a cylindrical barrel, with a connector integrated into the distal end for attachment of an infusion line set, and a movable plunger with an elastomer seal. The plunger is inserted into the open proximal end of the barrel to form a closed volume. To deliver drug, a mechanically driven piston is advanced forward, which in turn advances the cartridge plunger forward, reducing the internal volume of the cartridge, thus displacing fluid. Typically, the piston (part of the durable device) is not mechanically interlocked with the cartridge plunger because there is no need to retract the plunger once the cartridge has been filled and subsequently installed in the pump.

Because the pump piston is not interlocked with the cartridge plunger, there is a risk of unintentional delivery of drug if a positive pressure differential were to develop between the chamber that houses the reservoir and the external environment (location of infusion site). A positive pressure differential would impart a resultant force on the plunger which is directly proportional to the cross-sectional area of the drug reservoir's internal volume. If the resultant force exceeds the sustaining force of the cartridge plunger it will advance the plunger forward and thus deliver drug.

In one embodiment, the disclosed invention is a portable drug infusion pump with a mechanism for inhibiting the discharge of fluid from the pump's drug reservoir when the chamber containing the reservoir experiences a high pressure than the ambient environment. Most commonly this occurs in situations where the device is moved to locations where the atmospheric pressure is low, such as in an airplane, and drug is forced from the reservoir due to the pressure differential, e.g., the reservoir is at a higher pressure than ambient, thus causing the unintentional flow of drug out of the reservoir.

In an exemplary embodiment, the invention includes a drug reservoir with check valve. Typically, the reservoir is a syringe-like rigid cartridge for use in a portable drug infusion pump. The advantage of this novel design is that it prevents unintentional delivery of drug due to atmospheric pressure differentials or head height pressure differentials (i.e. siphoning). The check valve is in fluidic outlet path and requires a minimum positive pressure differential (cracking pressure) to open and allow flow of drug from the reservoir. The geometry of the check valve components determines the cracking pressure and can be adjusted to meet the functional requirements of the device in which it is to be used.

The device will typically include a housing containing a chamber. The chamber is configured to receive a cartridge containing a quantity of fluid. The fluid is typically a drug formulation, but on occasion may comprise saline or other material. At the interface between the chamber and the inserted cartridge is a preferred location for a check valve. The check valve may be constructed of a metal spring element in the shape of a disc. The geometry cut into the disc creates spring arms and a central surface that functions as a valve poppet. The valve seat may be constructed of an elastomer. The check valve described is the preferred embodiment, but the main elements of the check valve can be constructed in many configurations that produce the same functional results. For example, the elastomer valve seat could instead be constructed of a rigid material, and an elastomer disc could either be mechanically attached to the center of the metal spring element, or overmolded onto the metal spring element.

The check valve is installed inside the cartridge barrel at the bottom surface of the main bore and defines the interior volume of the reservoir. The check valve can be attached via a mechanical interference fit, ultrasonic weld, adhesive, or other standard methods of attachment typically used in disposable medical devices.

The check valve could be constructed of (5) components:

1) Substrate: Substrate would be an injection molded component made of a rigid polymer. Rigid polymer Substrate would support and orient check valve seat and support the perimeter one way valve. Rigid polymer Substrate would also define fluidic paths for both valves. Rigid polymer Substrate could also incorporate energy director geometries for attachment of check valve assembly to cartridge barrel of drug reservoir.

2) Check Valve Seat: An elastomer structure that could be overmolded onto the rigid polymer Substrate, or attached to the rigid polymer Substrate. The compliant elastomer structure functions as a check valve and works in conjunction with the Spring Element Disc.

3) Spring Element Disc: Spring Element Disc is a thin metal component of circular shape with geometry cut into it to form spring arms and a central poppet surface. Spring Element Disc could be photo-etched in a batch or continuous feed process to produce a precision geometry at minimal cost.

4) One Way Valve Flap: An elastomer structure that could also be overmolded onto the rigid polymer Substrate, or attached to the rigid polymer Substrate. The compliant elastomer structure would function as a one way valve in conjunction with mating surfaces of the Cartridge Barrel.

5) Cartridge Barrel: Bottom of Cartridge Barrel bore incorporates geometry that accepts and aligns the Spring Element Disc, and subsequently the overmolded Substrate (with overmolded or attached Check Valve Seat and One Way Valve Flap). The Substrate can then be attached (e.g. ultrasonically welded) to the Cartridge Barrel, trapping the Spring Element Disc between the Substrate and Cartridge Barrel. The Cartridge Barrel would also incorporate geometry at the bottom of its bore to act as a seat for the one way valve. Once assembled, both check valve and one way valve function are established.

Embodiments of the present invention allow end user to fill cartridge with drug in the same manner that a syringe would be filled. This is accomplished with a second one way-valve that is incorporated into the supporting structure of the check valve. When the plunger of the syringe-like cartridge is retracted, a negative pressure is created within the reservoir volume. This causes the one-way valve to open and allow drug to transfer from the vial to the reservoir. When the plunger is advanced forward the one-way valve closes and a positive pressure develops which is proportional to the force applied to the plunger. Once the internal positive pressure within the reservoir exceeds the cracking pressure of the check valve, the check valve opens and drug is dispensed. Typically this retracting and advancing motion is repeated several times during filling until all visible air is purged from the cartridge reservoir and the desired amount is transferred.

Check valve assembly disclosed is not limited to installation in the cartridge reservoir. It could be installed anywhere in the fluidic path between the drug reservoir and infusion site. Other viable locations for the check valve assembly are the proximal connector of the infusion line, the distal end of the infusion line, or anywhere in between. The check valve assembly could also be built into an independent adapter that could be placed between the cartridge reservoir and the infusion line.

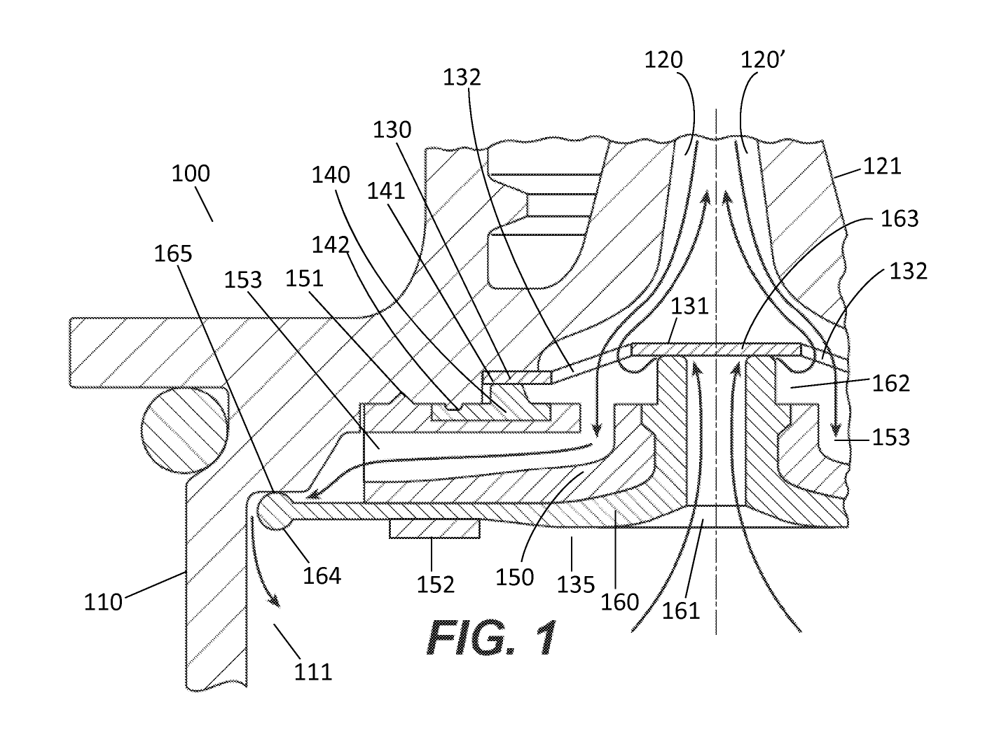

Turning to the specific features shown in the drawings figures, FIG. 1 illustrates an embodiment of the present invention. The drug delivery device 100 includes a drug reservoir 111 and check valve shown in its component parts. The check valve may comprise a valve body 135 that rests against the substrate 150 and a disc spring 130. The substrate may be secured to the luer 121 via a weld joint 151 that is created by, for example, an ultrasonic weld. The weld joint 151 may secure the substrate 150 to the luer by a variety of other manufacturing methods.

During drug delivery, an increase in pressure within the reservoir 111 biases the fluid from the reservoir against the poppet 131 of the disc spring 130. If sufficient pressure is used, also known as the cracking pressure, the spring arms 132 of the disc spring 130 will permit movement of the poppet 131 to allow fluid to pass from the reservoir 111 through outlet channel 161 and then delivered from the device 100 via the external fluid path 120, 120′ of the luer 121.

When there is no pressure biasing fluid out of the reservoir 111, the poppet 131 rests against the valve seat 162 with a bias provided by the spring arm 132 and a seal created by a compliant material that creates a dynamic seal 163. The valve body 135 may include a contact surface 152 as a point to transfer energy for welding and a dynamic outlet seal structure 160 which may be comprised of a compliant material. The periphery of the valve body 135 may also be configured with an inlet flap valve 164 to form the dynamic inlet seal 165 to inhibit the unintended ingress of fluid via inlet channel 153.

The disc spring 130 may be disposed within the luer 121 and configured to rest on a static seal structure 140 that is preferably made of a compliant or similar material to ensure against leakage of gas or fluid into or out of the reservoir. The static seal structure may include a static seal ridge 142 to provide a convenient site for bonding, welding, fusing, or otherwise attaching the static seal structure 140 to the luer 121. Further, the static seal structure 140 may include a protrusion or other suitable surface to form a disc spring support 141.

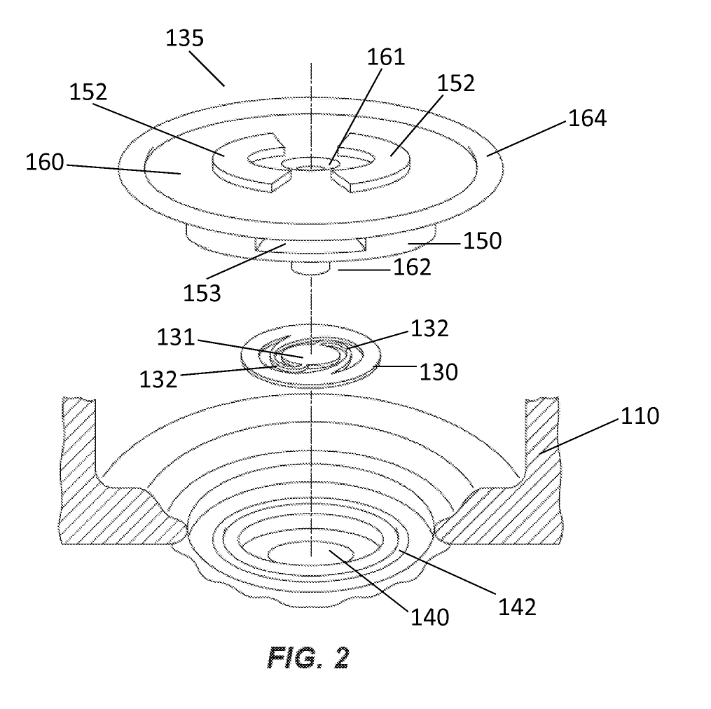

FIG. 2 illustrates an embodiment of the check valve of the present invention. Shown is the cartridge barrel 110 having a static seal structure 140 and a static seal ridge 142. A disc spring 130 is configured to be disposed on the disc spring support (not shown) and includes a poppet 131 that is moveably attached to the disc spring 130 by a spring arm 132. One or more spring arms 132 or equivalent structure may be used to permit biased movement of the poppet 131 to and away from the disc spring 130.

Illustratively, a compliant dynamic seal structure 160 having a valve seat 162 for contacting the poppet 131 is configured to mate with the cartridge barrel 110. The dynamic seal structure 160 may, on one side, have one or more contact surfaces 152 that may provide a site for welding during manufacturing and, on another side, include rigid substrate 150 in which the inlet channel 153 is formed. An outlet channel 161 can also be formed in the dynamic seal structure 160. The periphery of the dynamic seal structure 160 may also include an inlet valve flap 164 to ensure uniform contact between the dynamic seal structure of the valve 135 and the cartridge barrel 110.

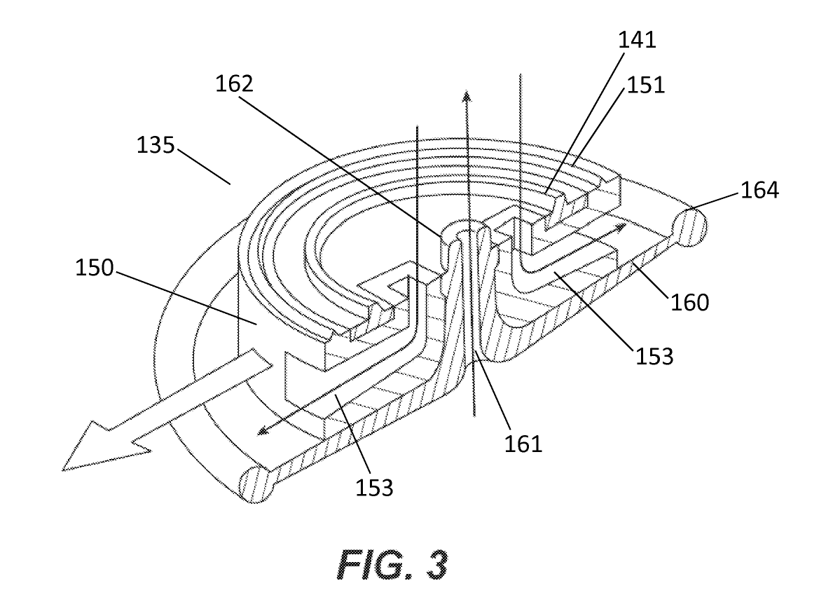

FIG. 3 further illustrates an embodiment of the valve 135 of the invention in which the substrate 150 is shown having the outlet channel 161 formed in its center and extending through a valve seat 162. Formed in the substrate 150 is also an inlet channel 153. One surface of the substrate 150 may include a disc spring support 141 and a weld joint 151.

It will be recognized that equivalent structures may be substituted for the structures illustrated and described herein and that the described embodiment of the invention is not the only structure, which may be employed to implement the claimed invention. In addition, it should be understood that every structure described above has a function and such structure can be referred to as a means for performing that function. While embodiments of the present invention have been shown and described herein, it will be obvious to those skilled in the art that such embodiments are provided by way of example only. Numerous variations, changes, and substitutions will now occur to those skilled in the art without departing from the invention.

It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A medical drug infusion device, comprising:
a housing having one or more chambers therein and configured to receive a cartridge; and
a check valve configured to minimize effluence of a fluid from the cartridge, wherein the check valve comprises a cartridge barrel having a barrel bore geometry that accepts and aligns with a spring element;
a substrate for defining a fluidic path and for engaging the barrel to hold a disc spring between the substrate and the cartridge barrel bore;
a one-way flap valve overmolded on the substrate; and
a check valve seat overmolded onto or as part of the substrate.

2. The medical infusion device of claim 1 wherein the check valve comprises a disc spring comprising a poppet and at least one spring arm.
